(19) United States
(12) Patent Application Publication
(10) Pub. No.: US 2012/0197083 A1
(43) Pub. Date: Aug. 2, 2012

(54) METHOD AND APPARATUS FOR ENDOSCOPE ADVANCEMENT

(75) Inventor: Benjamin Spenser, Bat Shlomo (IL)

(73) Assignee: GI MOTIONS LTD., Netanya (IL)

(21) Appl. No.: 13/499,698

(22) PCT Filed: Oct. 3, 2010
(86) PCT No.: PCT/IL2010/000802

§ 371 Date: Apr. 1, 2012

Related U.S. Application Data
(60) Provisional application No. 61/247,583, filed on Oct. 1, 2009.

(51) Int. Cl. A61B 1/01

(52) U.S. Cl. 600/115; 600/114

(57) ABSTRACT

An advancing mechanism for an endoscope constituted of: a generally tubular shaped sheath exhibiting an inner diameter and a length sufficient to at least partially encase an articulating section of the endoscope, the sheath comprising a plurality of deployable members arrayed thereabout, each exhibiting a first end attached to the sheath and a second end longitudinally extending away from the first end, each of the deployable members movable from a closed position to an open position, wherein in the closed position the deployable members do not substantially extend past an outer surface of the sheath and in the open position the second end of each of the deployable members extend past the outer surface of the sheath, and wherein in the open position each of the deployable members exhibit an angle in relation to the outer surface of the sheath of up to 60 degrees.

### TECHNICAL FIELD

The invention relates generally to the field of endoscopes, and in particular to a method and apparatus enabling advancement and retrieval of an endoscope through a body lumen.

### BACKGROUND

Diagnosis and treatment of gastro-intestinal (GI) diseases is advantageously performed responsive to visual inspection and occasion in-situ intervention or sampling. As a result, the field of endoscopy has advanced over the years, resulting in current solutions such as deep tube intubation and capsule endoscopy. Capsule endoscopy avoids a need to provide a tube extending from external of the subject to the target tissue, however control of capsule endoscopy is limited, and furthermore the ability to perform in-situ intervention or sampling is not provided.

An endoscope is typically provided with an articulation section near the distal tip of the endoscope tube, allowing the operator to articulate the distal tip in relation to a central axis of the endoscope tube by actuation of an articulation mechanism at the proximal end thereof. The articulation mechanism typically allows for articulation in any of 4 orthogonal directions, conventionally known as up, down left and right, typically defined in relation to the operator view from the camera at the endoscope tip. In reality each of the 4 orthogonal directions of articulation reflects bending of the articulation section from the center line axis of the elongated endoscope. One non-limiting example of an articulation mechanism is described in U.S. Pat. No. 5,704,898 issued Jan. 6, 1998 to Kokish, the entire contents of which is incorporated herein by reference.

One difficulty in tube based endoscopy is the need to control the advance of the distal tip of the endoscope through the tortuous area of the small intestine. One solution is the use of a balloon in cooperation with an overtube, wherein inflating the overtube balloon provides localized traction so that the endoscope disposed axially within the overtube may be advanced in relation thereto. Another solution is double balloon endoscopy, wherein inflatable balloons are provided at the distal tip of both the endoscope and the overtube. In addition to the ability to secure the overtube to the lumen wall, the endoscope may be fixed to the lumen wall by inflation of the endoscope tip balloon, thus allowing for advancing of the overtube. Upon completion of endoscopy, the endoscope is withdrawn from the patient by the operator.

Other methods include threaded catheters as described in U.S. Patent Application Publication US 2002/0045855 published Apr. 18, 2002 to Frassica, and a self propelled endoscope micro-robot as described in U.S. patent Ser. No. 6,702,734 issued Mar. 9, 2004 to Kim et al, the entire contents of each of which is incorporated herein by reference. Unfortunately each of these solutions adds complexity and some of the solutions are not completely compatible with commercially available endoscopes, or cannot overcome the above mentioned difficulties in advancement and retrieval.

It would thus be desirable to have an apparatus arranged for use with an endoscope which can provide an advancing mechanism, which further allows for retrieval.

### SUMMARY OF THE INVENTION

In view of the discussion provided above and other considerations, the present disclosure provides methods and apparatus to overcome some or all of the disadvantages of prior and present methods of endoscope advancement and retrieval. In an exemplary embodiment a sheath is provided covering at least a portion of an articulation section of an endoscope, the sheath comprising a plurality of deployable members, each of the deployable members exhibiting a closed position wherein the deployable members do not substantially extend past an outer surface of the sheath; and an open position wherein an end of each of the deployable members substantially extend from the sheath to exhibit an acute angle of up to 60° from the sheath. When the deployable members are in the second position, the endoscope is restricted from moving proximally, and thus advancing in relation to the body lumen responsive to articulation of the articulation section.

In one independent embodiment an advancing mechanism for an endoscope is provided, the advancing mechanism comprising: a generally tubular shaped sheath exhibiting an inner surface and a length configured and dimensioned to at least partially encase an articulating section of the endoscope, the sheath comprising a plurality of deployable members arrayed thereabout, each of the deployable members comprising a first end attached to the sheath and a second end longitudinally extending away from the first end, each of the deployable members movable from a closed position to an open position, wherein in the closed position the deployable members do not substantially extend past an outer surface of the sheath and in the open position the second end of each of the deployable members substantially extend past the outer surface of the sheath, and wherein in the open position each of the deployable members exhibit an angle in relation to the outer surface of the sheath of up to 60°, the angle determined from a proximal end of the sheath.

In one embodiment, in the open position, each of the deployable members exhibit an angle in relation to the outer surface of the sheath of up to 20°. In one further embodiment the deployable members each come to a tip at the second end thereof. In another further embodiment the sheath exhibits a plurality of perforations from the outer surface thereof through an inner surface thereof, each of the deployable members occupying a respective perforation when in the closed position.

In one further embodiment the advancing mechanism further comprises a remotely inflatable balloon disposed between the sheath and the articulating section of the endoscope, wherein the deployable members deploy to the open position responsive to inflation of the remotely inflatable balloon. In one yet further embodiment the deployable members are formed of a spring like material, the deployable members thereby urged to the closed position in the absence of inflation of the remotely inflatable balloon.

In another further embodiment the advancing mechanism further comprises an overtube, the overtube exhibiting an inner surface configured and dimensioned to at least partially encase the sheath and a length sufficient to cover all of the deployable members of the sheath. In one yet further embodiment, in the event that the overtube is advanced distally of the sheath, the deployable members deploy to the open position and in the event that the overtube is retarded proximally so as to cover the deployable members of the sheath the deployable members are retained to the closed position.

In another yet further embodiment the deployable members are formed of a spring like material, the deployable members thereby urged to the open position in the absence of the overtube. In one further embodiment the plurality of deployable members are arrayed along the length of the sheath and angularly displaced around the outer surface of the sheath.

Independently, a method of advancing an endoscope within a body lumen is provided, the method comprising: providing a generally tubular shaped sheath for an articulating section of the endoscope, the sheath exhibiting an inner surface and a length configured and dimensioned to at least partially encase the articulating section of the endoscope, the provided sheath comprising a plurality of deployable members arrayed thereabout, each of the deployable members comprising a first end attached to the provided sheath and a second end longitudinally extending away from the first end, each of the deployable members movable from a closed position to an open position; and opening each of the deployable members to exhibit an angle in relation to the outer surface of the provided sheath of up to 60°, the angle determined from a proximal end of the provided sheath, so as to provide advancement of the endoscope in relation to the body lumen responsive to articulating motion of the articulating section.

In one embodiment the method further comprises: providing an articulating motion of the articulating section. In one further embodiment the method further comprises: closing each of the deployable members so as to not substantially extend past an outer surface of the provided sheath, thereby enabling retrieval of the endoscope. In another embodiment, in the open position, each of the deployable members exhibits an angle in relation to the outer surface of the provided sheath of up to 20°.

In one embodiment the deployable members each come to a tip at the second end thereof In another embodiment the provided sheath exhibits a plurality of perforations from the outer surface thereof through an inner surface thereof, each of the deployable members occupying a respective perforation when in the closed position.

In one embodiment the method further comprises: providing a remotely inflatable balloon disposed between the provided sheath and the articulating section of the endoscope; and inflating the provided remotely inflatable balloon to deploy the deployable members deploy to the open position. In one further embodiment the deployable members are formed of a spring like material, the deployable members thereby urged to the closed position in the absence of the inflation of the remotely inflatable balloon.

In another embodiment the method further comprises: providing an overtube configured and dimensioned to at least partially encase the provided sheath and a length sufficient to cover all of the deployable members of the provided sheath; and advancing the provided overtube distally of the provided sheath so that the deployable members deploy to the open position. In one further embodiment the method further comprises: retarding the provided overtube so as to cover all of the deployable members of the provided sheath thus retaining the deployable members to the closed position.

In one embodiment the plurality of deployable members are arrayed along the length of the provided sheath and angularly displaced around the outer surface of the provided sheath.

Additional features and advantages of the invention will become apparent from the following drawings and description.

### BRIEF DESCRIPTION OF DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 illustrates a high level schematic diagram of an exemplary embodiment of an advancing mechanism for an endoscope, comprising a sheath in cooperation with a remotely inflatable balloon and an inflation mechanism for the remotely inflatable balloon;

FIG. 2 illustrates a high level schematic diagram of the sheath of FIG. 1 assembled on the endoscope of FIG. 1 to at least partially encase an articulating section of the endoscope;

FIG. 3A illustrates a perspective view of the sheath of FIG. 1 at least partially encasing the articulating section of the endoscope, with the plurality of deployable members in the closed position;

FIG. 3B illustrates a sectional view along a line A-A of FIG. 3A particularly illustrating the arrangement of the sheath, the inflatable balloon and the articulating section of the endoscope;

FIG. 3C illustrates a detailed view of a portion of the sheath of FIG. 3A, particularly illustrating the deployable members occupying a respective perforation when in the closed position;

FIG. 3D illustrates a frontal view of the sheath of the sheath of FIG. 3A, particularly illustrating the deployable members in the closed position;

FIG. 4A illustrates a perspective view of the sheath of FIG. 1 at least partially encasing the articulating section of the endoscope, with the plurality of deployable members in the open position;

FIG. 4B illustrates a sectional view along a line B-B of FIG. 4A particularly illustrating the arrangement of the sheath, the inflatable balloon and the articulating section of the endoscope;

FIG. 4C illustrates a detailed view of a portion of the sheath of FIG. 4A, particularly illustrating the deployable members extending past the outer surface of the sheath;

FIG. 4D illustrates a frontal view of the sheath of FIG. 4A, particularly illustrating the deployable members in the open position;

FIGS. 5A-5C illustrate various stages of advancement of the endoscope of FIGS. 1, 2 within a body lumen responsive to the sheath and to articulating motion of the articulating section;

FIGS. 6A-6B illustrate various stages of retrieval of the endoscope of FIGS. 1, 2 from within a body lumen responsive to deflation of the remotely inflatable balloon;

FIG. 7 illustrates a high level schematic diagram of the sheath of FIG. 1 assembled on the endoscope of FIG. 1 to at least partially encase an articulating section of the endoscope, and further comprising an overtube advanced distally of the sheath; and

FIGS. 8A-8B illustrate various stages of proximal retardation of the overtube of FIG. 7 so as to allow for retrieval of the endoscope of FIG. 8 from within a body lumen.

### DESCRIPTION OF EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

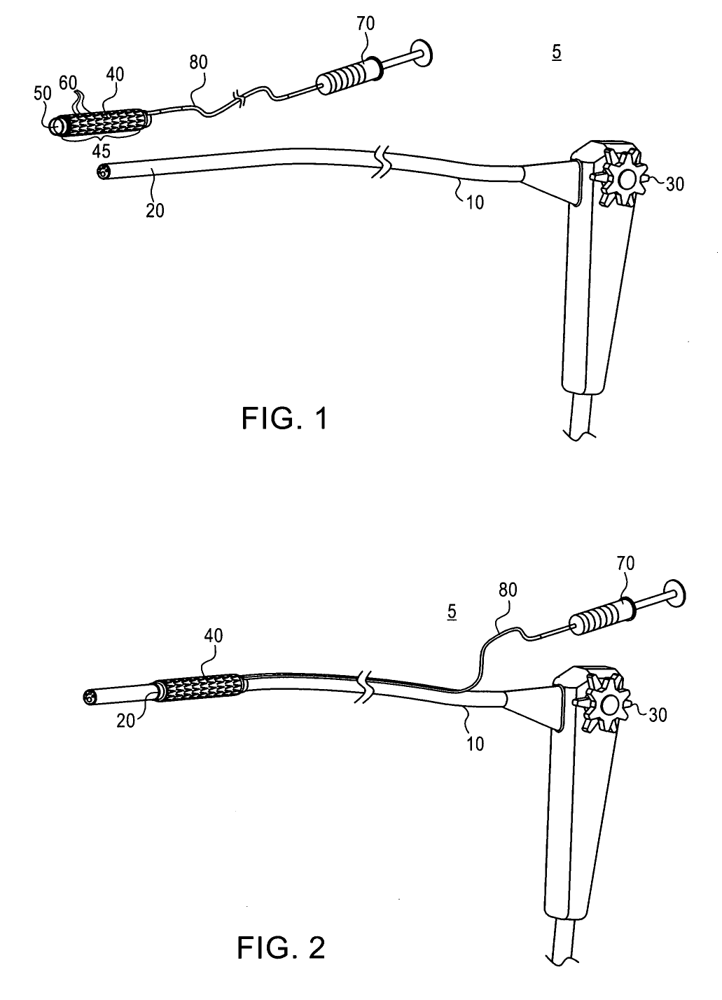

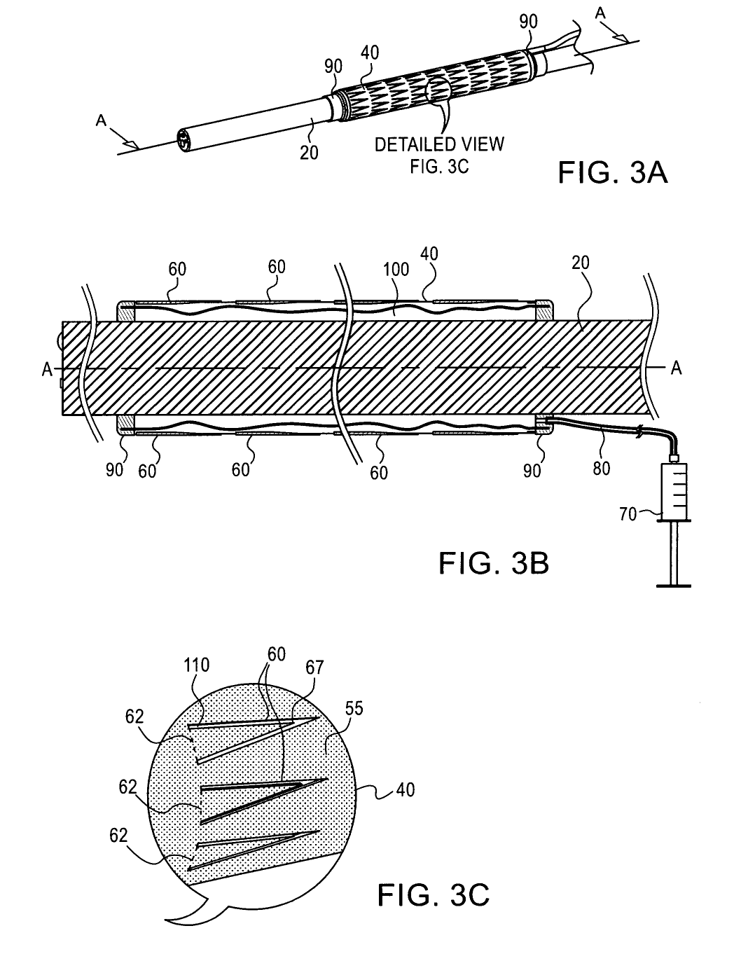

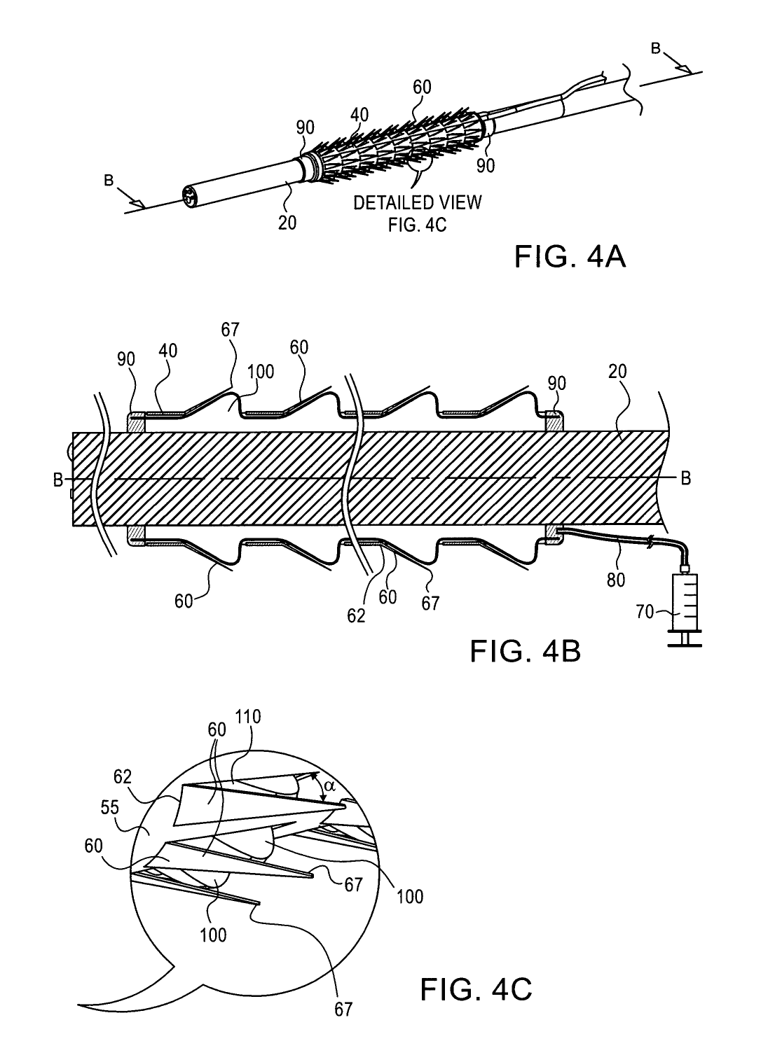

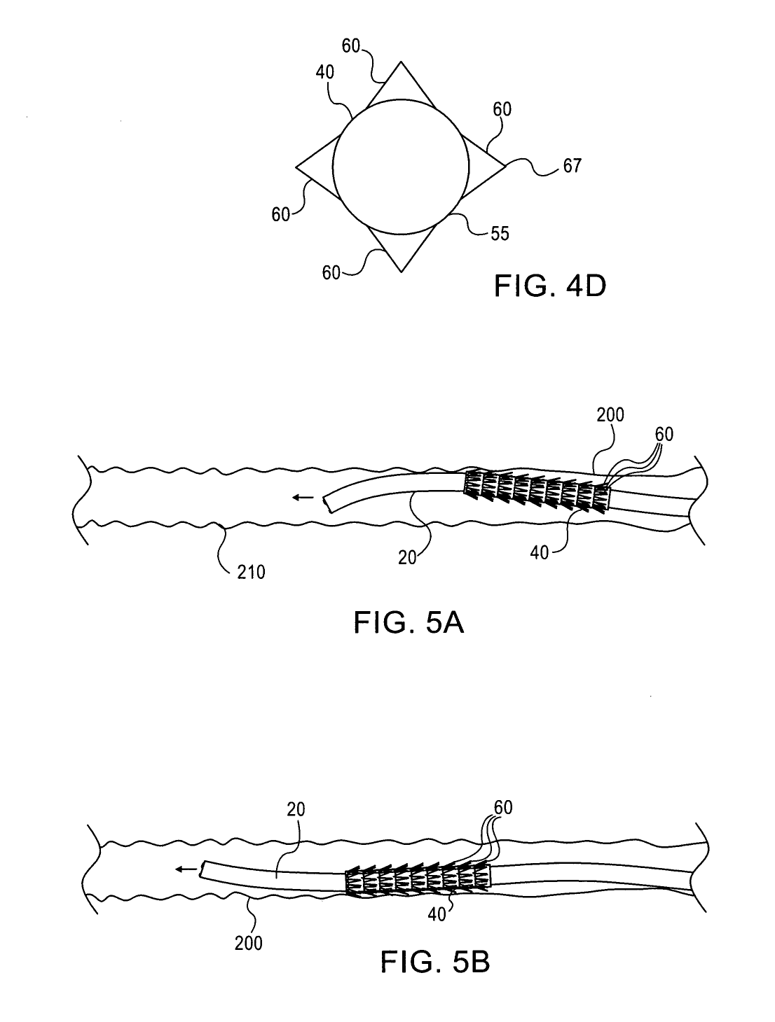

FIG. 1, 2, 3A-3D, and 4A-4D illustrate various views of an advancing mechanism 5 for use with an endoscope 10, the various views being taken together to described advancing mechanism 5. In particular, FIG. 1 illustrates a high level schematic diagram of an exemplary embodiment of advancing mechanism 5 for use with endoscope 10 comprising an articulating section 20 at a distal end of endoscope 10, articulating section 20 responsive to an articulating mechanism 30 located at a proximal end of endoscope 10, and further illustrating a sheath 40 exhibiting a length 45, an inner surface 50 and a plurality of deployable members 60. An inflation mechanism 70 in communication with sheath 40 via a tube 80 is further illustrated. FIG. 2 illustrates a high level schematic diagram of sheath 40 assembled on articulating section 20 of endoscope 10 of FIG. 1 to at least partially encase articulating section 20 of endoscope 10. FIG. 3A illustrates a perspective view of sheath 40 at least partially encasing articulating section 20 of endoscope 10, with deployable members 60 in a closed position. FIG. 3B illustrates a sectional view along a line A-A of FIG. 3A particularly illustrating a distal and a proximal locking ring 90 and a remotely inflatable balloon 100. FIG. 3C illustrates a detailed view of a portion of sheath 40 of FIG. 3A, particularly illustrating deployable members 60 each occupying a respective perforation 110 when in the closed position. Each deployable member 60 comprises a first end 62 hingeably connected to sheath 40 and a second end 67 longitudinally extending away from first end 62. In an exemplary embodiment second end 67 is a tip, or apex of a triangle, thus presenting a sharp point. FIG. 3D illustrates a frontal view of sheath 40 when deployable members 60 are in the closed position, illustrating that deployable members 60 do not extend beyond an outer surface 55 of sheath 40. FIG. 4A illustrates a perspective view of sheath 40 at least partially encasing articulating section 20 of endoscope 10, with deployable members 60 in the open position. FIG. 4B illustrates a sectional view along a line B-B of FIG. 4A particularly illustrating the arrangement of sheath 40, remotely inflatable balloon 100 when inflated, and articulating section 20. FIG. 4C illustrates a detailed view of a portion of sheath 40 of FIG. 4A, particularly illustrating deployable members 60 extending past outer surface 55 of sheath 40 and forming an angle α in relation to outer surface 55 of sheath 40. FIG. 4D illustrates a frontal view of sheath 40 when deployable members 60 are in the open position.

Articulating section 20 comprises a distal portion of endoscope 10, and in accordance with the prior art articulating section 20 may be articulated in any direction in relation to a center axis of articulating section 20, responsive to articulating mechanism 30, illustrated without limitation as a pair of knobs connected by wire filaments to distal endpoints of articulating section 20. The center axis of articulating section 20 is defined at a neutral position of articulating mechanism 30. Inner surface 50 of sheath 40 is configured and dimensioned to fit over a portion of articulating section 20 and length 45 of sheath 40 is configured and dimensioned to at least partially encase articulating section 20. There is no requirement that sheath 40 completely encase articulating section 20, and in particular length 45 may be less than the overall length of articulating section 20 without exceeding the scope. Sheath 40 is illustrated as a complete tubular shaped sheath, however this is not meant to be limiting in any way, and a split tubular shape or a partial enclosing generally tubular shape may be utilized without exceeding the scope.

Each deployable member 60 is preferably seated within a respective perforation 110 when in a closed position, each perforation 110 preferably proceeding from outer surface 55 to inner surface 50. Each deployable member 60 is hingeably connected to sheath 40 at first end 62, and the hingeable connection may be formed by scoring of the constituent material at first end 62. In an exemplary embodiment sheath 40 and deployable members 60 are formed of a single material and deployable members 60 are formed by laser cutting to define perforations 110. In one embodiment deployable members 60 are formed of a spring like material, such as Nitinol or other memory material, arranged to urge deployable members 60 to be in the closed position in the absence of any external force upon deployable members 60. A plurality of deployable members 60 are deployed, preferably arrayed along length 45 of sheath 40, and further preferably distributed angularly about outer surface 55. In one particular embodiment at least 4 deployable members 60 are evenly angularly distributed about outer surface 55, and a plurality of evenly angularly distributed deployable members 60 are arrayed about length 45 of sheath 40. In one further embodiment, as illustrated, deployable members 60 thus form a plurality of annular structures spaced longitudinally along length 45 of sheath 40. In another embodiment (not shown) a helical pattern, or a plurality of helical patterns, are formed by deployable members 60. As indicated above, in the closed position deployable members 60 do not appreciably extend beyond outer surface 55 of sheath 40, and in the open position deployable member 60 extend beyond outer surface 55 of sheath 40. In the open position deployable members 60 form angle α in relation to outer surface 55, angle α determined from a proximal end of sheath 40 being up to 60°, preferably up to 45° and further preferably up to 20°. In an exemplary embodiment angle α is in the range of 1°-20°. There is no requirement that deployable members 60 form a straight line from first end 62 to second end 67, and angle α is defined in relation to a line drawn from second end 67 to first end 62 in relation to outer surface 55 at the center line of first end 62.

Proximal and distal locking rings 90 secure the proximal end of sheath 40 and distal end of sheath 40, respectively, to articulating section 20 of endoscope 10. In an exemplary embodiment distal locking ring 90 further serves to secure the distal end of remotely inflatable balloon 100. In an exemplary embodiment proximal locking ring 90 is formed with a pass through for the distal end of tube 80, so as to allow bidirectional passage of fluid to and from remotely inflatable balloon 100 responsive to inflation mechanism 70. Remotely inflatable balloon 100 is preferably disposed between inner surface 50 of sheath 40 and an outer surface of articulation section 20 of endoscope 10.

Sheath 40 is slipped over the distal end of endoscope 10, and secured by proximal and distal locking rings 90 in place about articulating section 20. Remotely inflatable balloon 100 is inflated responsive to fluid pressure from inflation mechanism 70, and remotely inflatable balloon 100 forces deployable members 60 to the open position exhibiting angle α, with each deployable member 60 opening responsive to the hingeable connection of the respective first end 62. In an exemplary embodiment, deployable members 60 are sized such that second ends 67 meet the inner walls of a target body lumen, as will be described further.

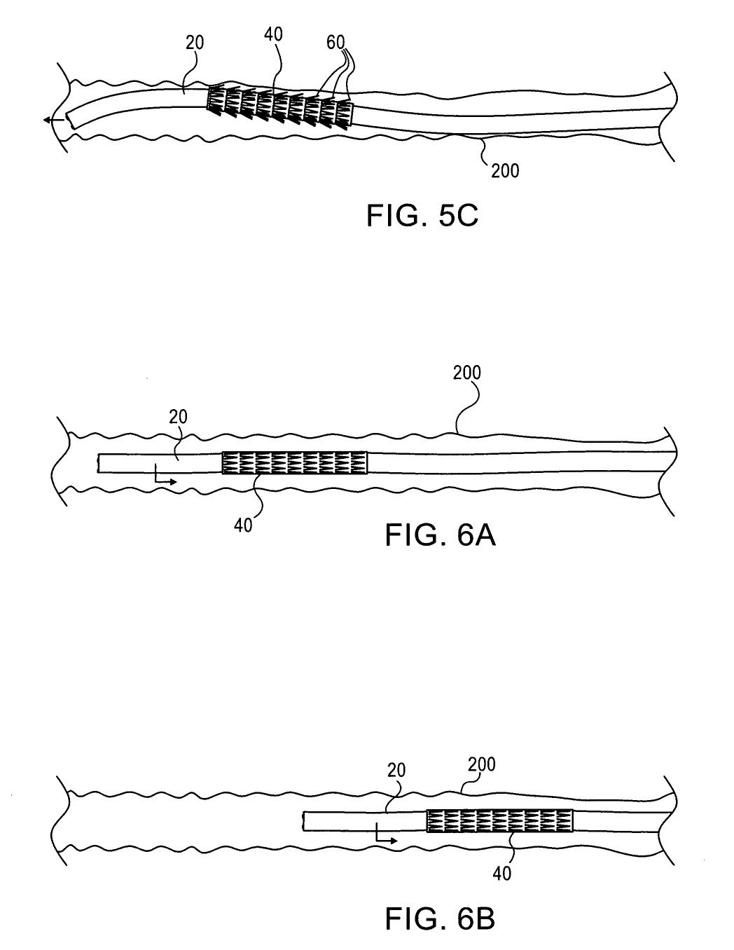

FIGS. 5A-5C illustrate various stages of advancement of endoscope 10 within a body lumen 200 responsive to sheath 40 of advancing mechanism 5. In particular, articulating section 20 is illustrated within body lumen 200, with deployable members 60 in the open position, and as indicated above extend to reach an inner wall 210 of body lumen 200, thus allowing for only a single direction of travel, as indicated by the arrow. Reverse direction is prevented by the action of second ends 67 meeting inner wall 210. Articulation of articulating section 20 responsive to articulating mechanism 30 is translated to an advancing motion since reverse motion is prevented. Acute angle α allows for consistent advance of articulating section 20. The term advance is not meant to exclusively mean absolute forward travel, and is particularly meant to include relative advancement along the body lumen. Thus, advancement of endoscope 10 is responsive to an articulating motion of articulating section 20 while reverse motion is prevented by sheath 40. Repeated articulating motion, or randomized motion, results in relative advancement.

FIGS. 6A-6B illustrate various stages of retrieval of endoscope 10 from within body lumen 200 responsive to deflation of remotely inflatable balloon 100. Responsive to inflation mechanism 70, remotely inflatable balloon 100 is deflated by removing fluid from remotely inflatable balloon 100, until deployable members 60 return to the closed position wherein they do not extend past outer surface 55 of sheath 40. As indicated above, preferably deployable members 60 are formed of a spring like material, and thus in the absence of force from remotely inflatable balloon 100 are urged to the closed position wherein they occupy the respective perforation 110. In the closed position deployable members 60 do not impede reverse travel of endoscope 10, and thus endoscope 10 may be withdrawn in accordance with prior art teachings, without limitation.

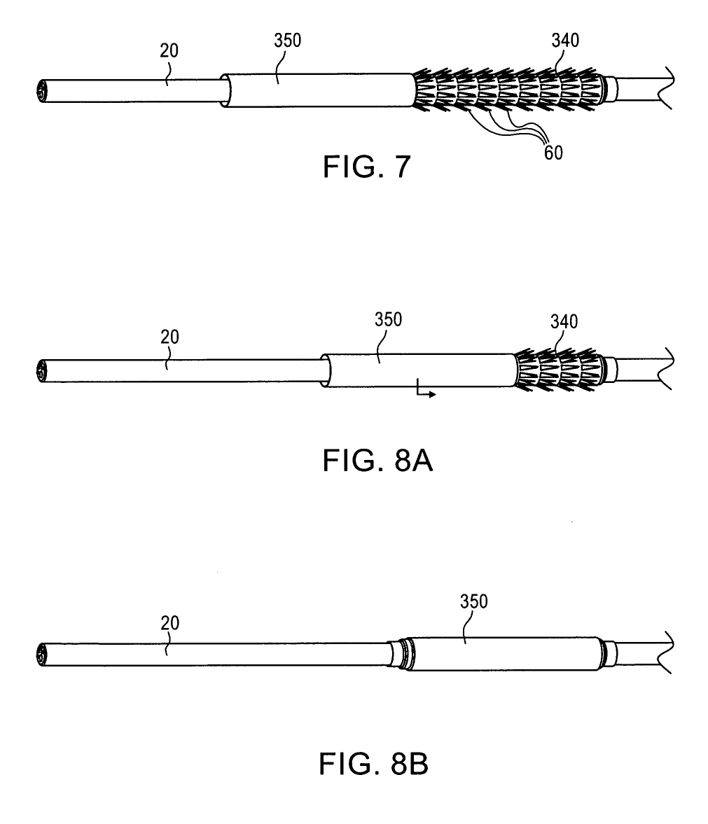

FIG. 7 illustrates a high level schematic diagram of a sheath 340 assembled on articulating section 20 of endoscope 10 to at least partially encase articulating section 20, and further comprising an overtube 350 advanced distally of sheath 340. Sheath 340 is in all respects similar to sheath 40 described above, with the exception that deployable members 60 of sheath 340 are arranged to urge towards the open position in the absence of any force. Overtube 350 exhibits an inner surface configured and dimensioned to at least partially encase sheath 340 and a length sufficient to cover all of the extendable members 60 of sheath 340. Overtube 350 may be advanced distally of sheath 340, thus allowing deployable members 60 to deploy to the open position or be retarded to encase all of the deployable members 60 thus placing them into the closed position. In such an embodiment the closed position is defined by the inner surface of overtube 350 and not by outer surface 55 of sheath 340. Overtube 350 may be advanced distally of sheath 340, or retarded over sheath 340 by a manipulation mechanism provided proximal of endoscope 10, as known to those skilled in the art.

FIGS. 8A-8B illustrate various stages of proximal retardation of the overtube 350 over sheath 340 so as to allow for retrieval of endoscope 10 from within a body lumen, such as body lumen 200 of FIGS. 6A-6B, since deployable members 60 are restrained into the closed position.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. An advancing mechanism for an endoscope, the advancing mechanism comprising:
a generally tubular shaped sheath exhibiting an inner surface and a length configured and dimensioned to at least partially encase an articulating section of the endoscope, said sheath comprising a plurality of deployable members arrayed thereabout, each of the deployable members comprising a first end attached to said sheath and a second end longitudinally extending away from the first end, each of the deployable members movable from a closed position to an open position,
wherein in the closed position the deployable members do not substantially extend past an outer surface of said sheath and in the open position the second end of each of the deployable members extend past the outer surface of said sheath, and
wherein in the open position each of the deployable members exhibit an angle in relation to the outer surface of said sheath of up to 60°, the angle determined from a proximal end of the sheath.

2. The advancing mechanism according to claim 1, wherein in the open position each of the deployable members exhibit an angle in relation to the outer surface of the sheath of up to 20°.

3. The advancing mechanism according to claim 1, wherein the deployable members each come to a tip at the second end thereof.

4. The advancing mechanism according to claim 1, wherein said sheath exhibits a plurality of perforations from the outer surface thereof through an inner surface thereof, each of the deployable members occupying a respective perforation when in the closed position.

5. The advancing mechanism according to claim 1, further comprising a remotely inflatable balloon disposed between said sheath and the articulating section of the endoscope, wherein the deployable members deploy to the open position responsive to inflation of the remotely inflatable balloon.

6. The advancing mechanism according to claim 5, wherein the deployable members are formed of a spring like material, the deployable members thereby urged to the closed position in the absence of inflation of the remotely inflatable balloon.

7. The advancing mechanism according to claim 1, further comprising an overtube, said overtube exhibiting an inner surface configured and dimensioned to at least partially encase said sheath and a length sufficient to cover all of the deployable members of said sheath.

8. The advancing mechanism according to claim 7, wherein in the event that said overtube is advanced distally of said sheath the deployable members deploy to the open position and in the event that said overtube is retarded proximally so as to cover the deployable members of said sheath the deployable members are retained to the closed position.

9. The advancing mechanism according to claim 8, wherein the deployable members are formed of a spring like material, the deployable members thereby urged to the open position in the absence of said overtube.

10. The advancing mechanism according to claim 1, wherein the plurality of deployable members are arrayed along the length of said sheath and angularly displaced around the outer surface of said sheath.

11. A method of advancing an endoscope within a body lumen, the method comprising:
providing a generally tubular shaped sheath for an articulating section of the endoscope, the sheath exhibiting an inner surface and a length configured and dimensioned to at least partially encase the articulating section of the endoscope, the provided sheath comprising a plurality of deployable members arrayed thereabout, each of the deployable members comprising a first end attached to the provided sheath and a second end longitudinally extending away from the first end, each of the deployable members movable from a closed position to an open position; and
opening each of the deployable members to exhibit an angle in relation to the outer surface of the provided sheath of up to 60°, the angle determined from a proximal end of the provided sheath, so as to provide advancement of the endoscope in relation to the body lumen responsive to articulating motion of the articulating section.

12. The method according to claim 11, further comprising:
providing an articulating motion of the articulating section.

13. The method according to claim 11, further comprising:
closing each of the deployable members so as to not substantially extend past an outer surface of the provided sheath, thereby enabling retrieval of the endoscope.

14. The method according to claim 11, wherein in the open position each of the deployable members exhibits an angle in relation to the outer surface of the provided sheath of up to 20°.

15. The method according to claim 11, wherein the deployable members each come to a tip at the second end thereof.

16. The method according to claim 11, wherein the provided sheath exhibits a plurality of perforations from the outer surface thereof through an inner surface thereof, each of the deployable members occupying a respective perforation when in the closed position.

17. The method according to claim 11, further comprising:
providing a remotely inflatable balloon disposed between the provided sheath and the articulating section of the endoscope; and
inflating the provided remotely inflatable balloon to deploy the deployable members deploy to the open position.

18. The method according to claim 17, wherein the deployable members are formed of a spring like material, the deployable members thereby urged to the closed position in the absence of the inflation of the remotely inflatable balloon.

19. The method according to claim 11, further comprising:
providing an overtube configured and dimensioned to at least partially encase the provided sheath and a length sufficient to cover all of the deployable members of the provided sheath; and
advancing the provided overtube distally of the provided sheath so that the deployable members deploy to the open position.

20. The method according to claim 19, further comprising:
retarding the provided overtube so as to cover all of the deployable members of the provided sheath thus retaining the deployable members to the closed position.

21. The method according to claim 11, wherein the plurality of deployable members are arrayed along the length of the provided sheath and angularly displaced around the outer surface of the provided sheath.
